(19) United States
(12) Patent Application Publication
(10) Pub. No.: US 2003/0212291 A1
(43) Pub. Date: Nov. 13, 2003

(54) Diamine curing agents for the preparation of polyurethane-urea elastomers

(75) Inventors: Vincent J. Gajewski, Cheshire, CT (US); Franklin H. Barrows, Waterbury, CT (US)

Correspondence Address: Michael P. Dilworth, Crompton Corporation, Benson Road, Middlebury, CT, 06749, US

(73) Assignee: CROMPTON CORPORATION

(21) Appl. No.: 10/139,024

(22) Filed: May 2, 2002

(51) Int. Cl. C07C 229/54

(52) U.S. Cl. 560/19

(57) ABSTRACT

Alkanediol ortho- and meta-diamino benzoates are prepared by reacting either ortho- or meta-amino benzoic acid esters with an alkanediol in the presence of a transesterification catalyst. The ortho-diamine may alternatively be prepared by reacting isatoic anhydride with the alkanediol. These alkanediol ortho- and meta-diamino benzoates are useful in the curing of urethane prepolymers to produce novel polyurethane urea elastomers.

### BACKGROUND OF THE INVENTION

1. Field of the Invention

The present invention relates to the preparation of alkanediol bis-(ortho- and meta-diamino benzoates). More particularly, the present invention relates to the preparation of:

A. alkanediol bis-(ortho- and meta-diamino benzoates) by reacting either ortho- or meta-amino benzoic acid esters with an alkanediol in the presence of a transesterification catalyst; or

B. alkanediol bis-(ortho-diamino benzoates) by reacting isatoic anhydride with an alkanediol in the presence of an esterification catalyst.

These diamines are useful for curing urethane prepolymers to produce polyurethane urea elastomers.

2. Description of Related Art

Alkanediol-p-diaminobenzoates have been used as curatives for preparing polyurethane ureas. These curatives can be prepared in a number of different processes.

U.S. Pat. No. 3,932,360 discloses the preparation of diamine cured polyurethane products by combining an isocyanate terminated urethane prepolymer with a compound of the formula:

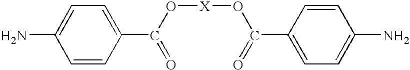

wherein X is a 2-12 carbon alkylene or cycloalkylene group.

U.S. Pat. No. 4,283,549 discloses a method of producing alkanediol-diaminobenzoates which includes esterifying nitro-benzoic acid and certain diols in a melt and then dissolving the intermediate in a solvent sparingly soluble in water such as an aromatic hydrocarbon, an ether or an ester and reducing with hydrogen gas.

U.S. Pat. No. 4,476,318 discloses a process for the preparation of 1,3-propanediol bis(p-aminobenzoate) comprising reacting a p-aminobenzoic acid alkali metal salt with dihalogenated propane in an aprotic polar solvent. In accordance with this process, diesterification is said to proceed under mild conditions without causing any undesirable side reactions and to provide 1,3-propanediol bis(p-aminobenzoate) in high purity and high yield.

U.S. Pat. No. 6,111,129 discloses a process for the direct preparation of alkanediol-diaminobenzoate comprising transesterifying an alkyl-p-aminobenzoate with a diol in the presence of a transesterification catalyst.

EP 0 677 542 A2 discloses polyurethane-urea elastomers that are made using as a chain extender 2-methyl-1,3-propanediol-bis-p-aminobenzoate, which is the reduction product from hydrogenating 2-methyl-1,3-propanediol-bis-p-nitrobenzoate. The latter composition is preferably made by esterifying p-nitrobenzoic acid and 2-methyl-1,3-propanediol using a stoichiometric excess of the diol initially, adequate to render the reaction mixture processible, and after converting to a nonvolatile form sufficient diol to form diester with substantially all of the acid, removing free diol by distillation while continuing the esterification of unreacted acid and transesterification of the monoester formed to diester. This process, which is said to be applicable broadly to esterification of other nitroaromatic acids with other aliphatic diols, is said to produce high yields of diester without needing extraneous solvent for processibility and with only water as a by-product. The 2-methyl-1,3-propanediol-bis-p-aminobenzoate exhibits reactivity and processing characteristics which are said to make it a suitable drop-in replacement for MOCA in polyurethane-urea elastomer manufacture.

The curatives provided by all the above processes are the para-amino benzoic esters of diols. The primary objective of the para isomers is to replace methylene bis orthochloroaniline (MOCA), which has been on the lists of suspect carcinogens of various regulatory agencies.

Japanese Kokai 61012653 A2 discloses the manufacture of aminobenzoic acids by the reduction of nitrobenzoic acids in a water-immiscible hydrocarbon solvent in the presence of sulfur or sulfur-containing compounds and Raney nickel or sulfur-poisoned Raney nickel.

Japanese Kokai 58059949 A2 discloses the preparation of 1,3-propanediol-bis(p-aminobenzoate) by heating Cl(CH₂)₃R(R=Cl, Br) with sodium or potassium p-aminobenzoate in an aprotic dipolar solvent, e.g., dimethyl formamide, dimethyl sulfoxide, or N-methylpyrrolidone. Thus, 0.15 mol of Cl(CH₂)₃Cl was heated with 0.3 mol sodium p-aminobenzoate in dimethyl sulfoxide at 100° for three hours to give 91.5 percent 1,3-propanediol bis(p-aminobenzoate) of 97.4 percent purity as opposed to a 3 percent yield with ethylene glycol as solvent. Cl(CH₂)ₙCl(n=2, 4, 5, 8), instead of Cl(CH₂)₃Cl gave the corresponding alkylene bis(p-aminobenzoates) in much lower yield.

Japanese Kokai 57120555 A2 discloses the preparation of compounds of the structure (H₂NC₆H₄COO)₂X, where X=(substituted) alkylene, by esterification of the benzoic acid with a dihaloalkane in the presence of a quaternary ammonium salt.

German OLS 2902740 discloses the preparation of alkanediol-bis(aminobenzoic acid esters) by esterifying nitrobenzoates with diols HO(CH₂)ₙOH(n=2, 3, 5, 6) in the presence of an aromatic sulfonic acid catalyst and reducing the resulting nitro compounds.

Although the para isomers of alkanediol benzoates make good MOCA-like polyurethane ureas, they do have certain drawbacks. They are high melting and therefore difficult to process; they do not readily produce softer elastomers, i.e., below 70 Shore A hardness; and they have very fast reaction times with certain isocyanates, such as methylenediphenyidiisocyanate (MDI) and p-phenylenediisocyanate (PPDI). It would be advantageous to develop a diaminobenzoate free from these disadvantages.

### SUMMARY OF THE INVENTION

The present invention is directed toward the preparation of alkanediol bis(ortho-diaminobenzoates) and alkanediol bis(meta-diaminobenzoates) and the polyurethane ureas derived therefrom. Any of the above methods may be used to prepare the alkanediol ortho- and meta-isomers of benzoic acid, but the preferred method is a one-step process comprising transesterifying an aminobenzoic ester with a diol, with the release of an alcohol.

More particularly, the present invention is directed to a process for the direct preparation of alkanediol ortho- or meta-diaminobenzoates comprising transesterifying an alkyl ortho- or meta-aminobenzoate with an alkanediol in the presence of a transesterification catalyst.

In another aspect, the present invention is directed to a process for the direct preparation of alkanediol ortho-diaminobenzoates comprising esterifying isatoic anhydride with an alkanediol in the presence of an esterification catalyst, wherein the molar ratio of anhydride to alkanediol is at least 2:1.

In still another aspect, the present invention is directed to a polyurethane urea elastomer prepared by curing a urethane prepolymer with an alkanediol ortho- or meta-diaminobenzoate prepared by a process comprising transesterifying an alkyl ortho- or meta-aminobenzoate with an alkanediol in the presence of a transesterification catalyst.

### DESCRIPTION OF THE PREFERRED EMBODIMENTS

According to the present invention, alkanediol ortho- or meta-aminobenzoates are prepared with a diol in the presence of a transesterification catalyst, according to the equations:

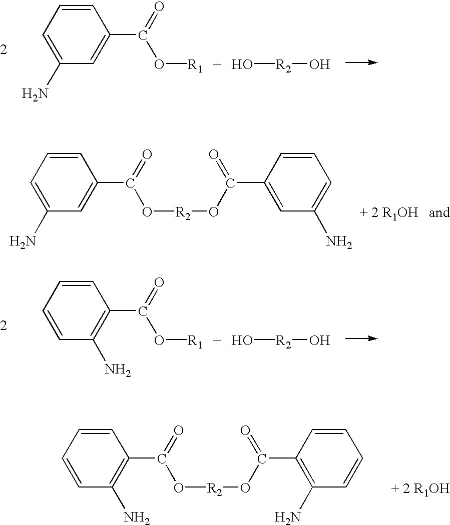

wherein:

R₁

is an alkyl group, which may be unsubstituted or substituted with non-interfering moieties and which includes cyclic alkyl groups. R₁ is preferably a linear or branched alkyl group of from 1 to 12 carbon atoms, e.g., methyl, ethyl, propyl, butyl, pentyl, hexyl, heptyl, octyl, nonyl, decyl, undecyl, dodecyl, and isomers thereof. R₁ is more preferably an alkyl group of from 1 to 4 carbon atoms and, most preferably, is ethyl; and

R₂ is an alkylene group, which may be unsubstituted or substituted with non-interfering moieties and which includes cyclic alkylene groups. R₂ is preferably a linear or branched alkylene group of from 1 to 12 carbon atoms, e.g., methylene, ethylene, propylene, butylene, pentylene, hexylene, heptylene, octylene, nonylene, decylene, undecylene, dodecylene, and isomers thereof. R₂ is more preferably an alkylene moiety from 1 to 4 carbon atoms and, most preferably, propylene, i.e., —CH₂CH₂CH₂— or the isomer —CH(CH₃)—CH₂—.

Those skilled in the art will readily recognize that isatoic anhydride, i.e.,

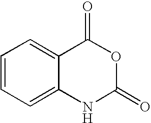

could be substituted for the ortho-aminobenzoate in the second of the above equations.

The following diols are exemplary of those that can be used in the reaction of the present invention:

1,3-butanediol,

1,4-butanediol,

2,3-butanediol,

2-n-butyl-2-ethyl-1,3-propanediol,

3-chloro-1,2-propanediol,

1,4-cyclohexanediol,

2,5-dimethyl-2,5-hexanediol,

2,2-dimethyl-1,3-propanediol,

2,2-diphenyl-1,3-propanediol,

1,12-dodecanediol,

ethylene glycol,

2-ethyl-1,3-hexanediol,

2-ethyl-2-methyl-1,3-propanediol,

1,7-heptanediol,

1,6-hexanediol,

2,5-hexanediol,

2-methyl-1,4-butanediol,

2-methyl-2,4-pentanediol,

2-methyl-1,3-propanediol,

1,8-octanediol,

1,5-pentanediol,

2,4-pentanediol,

1-phenyl-1,2-ethanediol,

1,2-propanediol,

1,3-propanediol,

1,1,4,4-tetraphenyl-1,4-butanediol, and

2,2,4,4-tetramethyl-1,3-cyclobutanediol.

A preferred diol is 1,3-propanediol.

Any transesterification catalyst can be used, but tin compounds, especially dibutyltindiacetate, are preferred. An exemplary listing of catalysts that can be employed in the practice of the present invention includes stannous octoate, stannous oxalate, dibutyltindilaurate, dioctyltindilaurate, dibutyltindi-2-ethylhexoate, tetraisopropyl titanate, tetrabutyl titanate, tetrakis-2-ethylhexyl titanate, dibutyltindilauryl mercaptide, dibutyltindiisooctylmercapto acetate, dioctyltindilauryl mercaptide, dimethyltindilauryl mercaptide, dimethyltindiisooctylmercapto acetate, dibutyltindilaurate, dibutyltin oxide, butyl stannoic acid, and the like.

The alkylaminobenzoate to diol ratio can be stoichiometric, i.e., 2:1,but the use of an excess of alkylaminobenzoate, e.g., in the range of greater than 2:1 up to about 5:1,preferably from greater than 2:1 up to about 3:1,is permissible and, in certain cases, may be found to be advantageous.

The reaction can be carried out in a temperature range of from about 100° C. to about 180°0 C., but a range of from about 110° C. to about 160° C. is preferred, and from about 120° C. to about 140° C. most preferred.

Once obtained, the product can be easily purified via crystallization from alcohol, preferably an alcohol having from one to eight, more preferably, from one to four carbon atoms, e.g. methanol, ethanol, propanol, butanol, pentanol, hexanol, heptanol, octanol, and the like, and isomers thereof.

The crystallization solvent can be readily recycled. The filtrate bottoms that contain any excess alkylaminobenzoate, monoester and/or catalyst can be recycled to the next batch without detriment, making the process of the present invention one that is economically efficient and environmentally beneficial.

The alkanediol-diaminobenzoate curing agents prepared by the process of the present invention are capable of curing isocyanate terminated urethane prepolymers to provide strong, rubbery, abrasion-resistant solids in conventional hot molding or in solvent based coating systems.

For solvent-based systems the curing agents are soluble in several conventional solvents used for coating techniques and possess kinetics that provide useful working times and conditions and also provide the desired three-dimensional structure in the cured polyurethanes.

In conventional hot molding techniques the curing agents prepared by the process of the present invention possess melting points generally within the desired range for such techniques and a reasonable degree of supercooling properties, are compatible with a wide range of isocyanate terminated urethane prepolymers, and, in general, exhibit kinetics that provide useful pot lives and curing times. In addition, the curing agents are generally stable to decomposition at their melting points.

Suitable isocyanate terminated urethane prepolymers for use with the curing agents prepared by the process of the present invention are known in the art and are disclosed, for example, in Advances in Urethane Science and Technology, Vol. 1,K. D. Frisch and S. L. Regan (Technomic Publishing Company, New Jersey, 1971) and Polyurethane Coatings, Keith Johnson (Noyes Data Corporation, New Jersey, 1972). Such isocyanate terminated prepolymers include those generally designated in the art as “polyester” and “polyether” types. Examples of preferred isocyanate terminated urethane prepolymers are disclosed in U.S. Pat. No. 3,188,302,incorporated herein by reference.

The curing agents prepared by the process of the present invention can be employed singly or in a combination of two or more in order to obtain the optimum properties of each curing agent. It should also be understood that conventional additives can be employed in the prepolymer/curing agent composition, such as, catalysts, fillers, plasticizers, and the like.

Polyurethane products cured using the curing agents prepared by the process of the present invention can be employed wherever conventional polyurethanes are employed. For example, they are particularly useful for machine parts, potting and encapsulation of electronic equipment, and as a metal replacement. The specific properties of such products will depend upon the specific isocyanate terminated prepolymer, the specific diamine curing agent, the ratio of isocyanate to amine, and the curing cycle employed.

The curing agents prepared by the process of the present invention are satisfactorily employed in curing isocyanate terminated urethane prepolymers when used at a ratio of about 0.5:1 to about 2:1 based on moles of amine to moles of isocyanate and, more preferably, at a ratio of about 0.8:1 to about 1.2:1.

Examples V and VI of U.S. Pat. No. 3,932,360 provide useful descriptions, respectively, of the diamine curing of an isocyanate terminated urethane prepolymer by the hot melt technique and by the solvent method.

According to the first of those disclosures, 10 grams of a commercially available isocyanate terminated prepolymer containing 4 percent by weight of available NCO groups and prepared from polytetramethylene ether glycol and 2,4-toluene diisocyanate (ADIPRENE L-100,E. I. Crompton Corporation) is heated in a glass vessel to 110° C. Then, 1.4 grams of 1,3-propanediol di-p-aminobenzoate is melted and heated to 150° C. The diamine is then added to the prepolymer, with thorough stirring. The mixture is then degassed and poured into a metal mold preheated to 110° C. The mold and its contents are maintained at 110° C. for three hours, whereby a tough, cured polyurethane elastomer is obtained upon removal therefrom.

According to the second of the disclosures, 1.3 grams of 1,3-propanediol di-p-aminobenzoate is dissolved in 10 grams of 2-ethoxyethyl acetate with slight warming, and the thus formed solution is added to 10 grams of ADIPRENE L-100 and stirred thoroughly. The viscosity stability of the resulting solution is suitable for most applications. The solution is applied to glass and metal surfaces by spraying, dipping, and draw-down techniques and cured at 110° C. for three hours, whereby tough elastomeric films are obtained.

Various features and aspects of the present invention are illustrated further in the examples that follow. While these examples are presented to show one skilled in the art how to operate within the scope of the invention, they are not intended in any way to serve as a limitation upon the scope of the invention.

### EXAMPLES

### Example 1

1,3-Propanediol Bis-(2-aminobenzoate)

In a 500 mL four-necked, round bottom flask equipped with a thermocouple, a mechanical stirrer, a distillation head, and a subsurface nitrogen sweep were placed 123.9 grams (0.75 mole) of ethyl 2-aminobenzoate, 22.93 grams (0.3 mole) of 1,3-propanediol, and 3.42 grams of dibutyltindiacetate. Over a period of one-half hour the reaction mix was heated to 160° C. as the ethanol produced by the reaction was distilled off. The reaction was followed by high performance liquid chromatography by observing the disappearance of the starting ethyl 2-amino benzoate and the conversion of the intermediate monoester to the diester. The reaction mixture was added to 400 grams of isopropanol and on cooling the title compound precipitated and was isolated by filtration. The filter cake was washed with isopropanol and dried to a constant weight of 74.5 grams (79.1% yield). The melting point of the product was 78 to 81° C., and the relative area HPLC analysis of the product showed it to be 93.5% pure. The infrared spectrum was consistent with the structure.

### Example 2

1,3Propanediol Bis-(3-aminobenzoate)

In a 100 mL round bottom flask equipped with a thermometer, a mechanical stirrer, a distillation head, and a subsurface nitrogen sweep were placed 62 grams (0.375 mole) of ethyl 3-aminobenzoate, 11.4 grams (0.15 mole) of 1,3propanediol and 1.7 grams of dibutyltindiacetate. Using the same procedure as in Example 1, 38.6 grams (82% yield) of the compound was recovered. A portion was re-crystallized from isopropanol to give a product having a melting point of 116-118° C. and an HPLC analysis of 94.3%. The infrared spectrum was consistent with the structure.

### Example 3

Polyurethane Urea from 1,3-Propanediol Bis-(2-aminobenzoate)

A quantity of 22.4 grams of 1,3-propanediol bis-(2-aminobenzoate) prepared as in Example I was heated to melt and mixed into 100 grams of Vibrathane B625,an MDI/PTMG prepolymer with a 6.3% residual NCO. The mixture was degassed for approximately one minute and then cast into a metal mold at 115° C. The sheets were post-cured for 16 hours at 115° C.

### Example 4

Polyurethane Urea from 1,3-Propanediol Bis-(2-aminobenzoate)

A quantity of 15.3 grams of 1,3-propanediol bis-(2-aminobenzoate) prepared as in Example I was heated to melt and mixed into 100 grams of Adiprene L-100,a TDI/PTMG prepolymer with a 4.3% residual NCO. The mixture was degassed for approximately one minute and cast into a 100° C. mold. The sheets were post-cured 16 hours at 100° C.

### Comparative Example A

60 A Polyether Polyurethane

3.5 grams of 1,4 butanediol and 0.8 gram of trimethylolpropane were premixed, heated to melt, and mixed into 100 grams of Adiprene L-100with a 4.2% residual NCO. The mixture was degassed for approximately one minute and cast into metal molds at 100° C. The sheets were post-cured 16 hours at 100° C.

The physical properties of the products of Examples 3 and 4 and Comparative Example A are shown in Table 1.

TABLE 1

| EXAMPLE | 3 | 4 | A |
|---|---|---|---|
| DUROMETER SHORE A | 65 | 55 | 60 |
| 100% MODULUS | 300 PSI | 200 PSI | 275 PSI |
| 300% MODULUS | 625 PSI | 300 PSI | 500 PSI |
| TENSILE STRENGTH | 2100 PSI | 1800 PSI | 2100 PSI |
| ELONGATION | 475% | 625% | 500% |
| TEAR STRENGTH pli | 120 | 160 | 90 |
| SPLIT TEAR pli | 45 | 32 | 12 |

It can be seen from the above examples that the aminobenzoates of the present invention produce polyurethanes having low durometer hardness while retaining high tear strength.

In view of the many changes and modifications that can be made without departing from principles underlying the invention, reference should be made to the appended claims for an understanding of the scope of the protection to be afforded the invention.

What is claimed is:

1. A process for the direct preparation of alkanediol ortho- or meta-diaminobenzoates comprising transesterifying an alkyl ortho- or meta-aminobenzoate with an alkanediol in the presence of a transesterification catalyst.

2. A process for the preparation of alkanediol ortho- or meta-diaminobenzoates comprising reacting an ortho- or para-aminobenzoic acid ester with an alkanediol in the presence of a transesterification catalyst, according to one of the following equations: 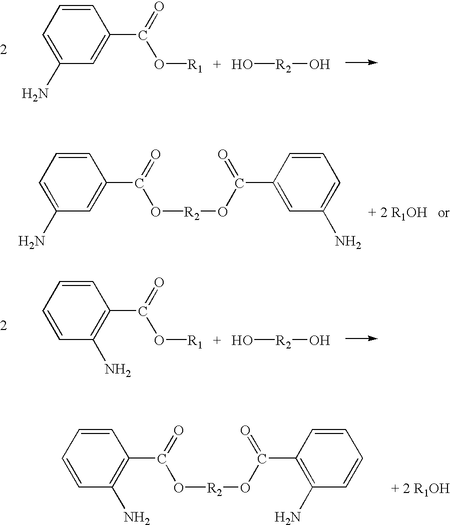
wherein:
R₁ is an alkyl group from 1 to 12 carbon atoms and R₂ is an alkylene moiety from 1 to 12 carbon atoms.

3. The process of claim 2 wherein R₁is a straight-chain or branched alkyl moiety of 1 to 12 carbon atoms.

4. The process of claim 2 wherein R₁ is ethyl.

5. The process of claim 2 wherein R₂ is a substituted or unsubstituted alkylene or cycloalkylene moiety of 1 to 12 carbon atoms.

6. The process of claim 5 wherein R₂ is a substituted alkylene moiety.

7. The process of claim 2 wherein the diol is selected from the group consisting of:
1,3-butanediol,
1,4-butanediol,
2,3-butanediol,
2-n-butyl-2-ethyl-1,3-propanediol,
3-chloro-1,2-propanediol,
1,4-cyclohexanediol,
2,5-dimethyl-2,5-hexanediol,
2,2-dimethyl-1,3-propanediol,
2,2-diphenyl-1,3-propanediol,
1,12-dodecanediol,
ethylene glycol,
2-ethyl-1,3-hexanediol,
4 2-ethyl-2-methyl-1,3-propanediol,
5 1,7-heptanediol,
1,6-hexanediol,
2,5-hexanediol,
2-methyl-1,4-butanediol,
2-methyl-2,4-pentanediol,
2-methyl-1,3-propanediol,
1,8-octanediol,
1,5-pentanediol,
2,4-pentanediol,
1-phenyl-1,2-ethanediol,
1,2-propanediol,
1,3-propanediol,
1,1,4,4-tetraphenyl-1,4-butanediol, and
2,2,4,4-tetramethyl-1,3-cyclobutanediol.

8. The process of claim 7 wherein the diol is 1,3-propanediol.

9. The process of claim 2 wherein the transesterification catalyst is a tin compound.

10. The process of claim 9 wherein the transesterification catalyst is dibutyltindiacetate.

11. The process of claim 2 wherein the reaction is carried out at a temperature in the range of about 100° C. to about 180° C.

12. The process of claim 2 further comprising the step of purifying the alkanediol-diaminobenzoate by crystallization from alcohol.

13. The process of claim 12 wherein the alcohol is one having from one to eight carbon atoms.

14. The process of claim 11 further comprising the step of purifying the alkanediol-diaminobenzoate by crystallization from alcohol.

15. The process of claim 14 wherein the alcohol is one having from one to eight carbon atoms.

16. A process for the direct preparation of alkanediol ortho-diaminobenzoates comprising esterifying isatoic anhydride with an alkanediol in the presence of an esterification catalyst, wherein the molar ratio of anhydride to alkanediol is at least 2:1.

17. A polyurethane urea elastomer prepared by curing a urethane prepolymer with an alkanediol ortho- or meta-diaminobenzoate prepared by a process comprising transesterifying an alkyl ortho- or meta-aminobenzoate with an alkanediol in the presence of a transesterification catalyst.

18. A polyurethane urea elastomer prepared by curing a urethane prepolymer with an alkanediol ortho-diaminobenzoate prepared by a process comprising comprising esterifying isatoic anhydride with an alkanediol in the presence of an esterification catalyst, wherein the molar ratio of anhydride to alkanediol is at least 2:1.
